(19) United States
(12) Patent Application Publication
(10) Pub. No.: US 2022/0354357 A1
(43) Pub. Date: Nov. 10, 2022

(54) SYSTEMS AND APPARATUSES FOR PERFORMING AN ENDOSCOPY WITH A DETACHABLE INSERTION CORD

(71) Applicant: The Board of Regents of The University of Texas System, Austin, TX (US)

(72) Inventors: Cristhiaan D. Ochoa-Arenas, Dallas, TX (US); Matthew J. Leveno, Dallas, TX (US); Manogna Yanamala, Dallas, TX (US); Kaylen Morrison, Dallas, TX (US); Ricardo Peralta, Dallas, TX (US); Micah Cox, Dallas, TX (US); Abhishek Srinivas, Dallas, TX (US); Mia Spennato, Dallas, TX (US)

(21) Appl. No.: 17/738,750

(22) Filed: May 6, 2022

Related U.S. Application Data
(60) Provisional application No. 63/185,277, filed on May 6, 2021.

(51) Int. Cl. A61B 1/267; A61B 1/00; A61B 1/05

(52) U.S. Cl. CPC A61B 1/2676; A61B 1/00114; A61B 1/00124; A61B 1/00128; A61B 1/05; A61B 1/00066

(57) ABSTRACT

An endoscope (e.g., bronchoscope) includes an insertion cord detachable from a housing. A first working channel connector at the housing may form a first detachable connection with a second working channel connector at a detachable end of the insertion cord, a first electrical connector at the housing may form a second detachable connection with a second electrical connector at the detachable end, and a first mechanical working pulley connector at the housing may form a third detachable connection with a second working pulley connector. The first working channel connector, the first electrical connector, and the first mechanical pulley connector may be formed into a first half of an integrated connector. The second working channel connector, the second electrical connector, and the second mechanical pulley connector may be formed into a second half of the integrated connector to mate with the first half with one or more engagement action.

### CROSS-REFERENCE TO RELATED APPLICATIONS

The present application claims priority under 35 U.S.C. § 119 to U.S. Provisional Application No. 63/185,277 entitled “Systems and Apparatuses for Performing an Endoscopy with a Detachable Insertion Cord” and filed on May 6, 2021, which is specifically incorporated by reference herein in its entirety.

### BACKGROUND

### 1. Field

The present disclosure relates generally to systems, methods, and apparatuses for performing an endoscopy. In at least one example, the present disclosure relates to a bronchoscope with a detachable insertion cord that enables a single operator to detach the insertion cord during operation to perform an intubation.

### 2. Discussion of Related Art

Bronchoscopes are often used for intubating patients in the emergency room with acute breathing difficulties. Typical bronchoscope designs have a flexible bronchoscope insertion cord attached to a large, bulky handle. This makes endotracheal tube insertion difficult because in order to intubate a patient during a bronchoscopy the bronchoscope must have the endotracheal tube on the insertion cord before it is inserted, which makes the clearance between a patient's airways and the insertion cord very small while it is inserted. Furthermore, an operator may not know if an endotracheal tube will be needed prior to inserting the insertion cord into the patient and so, out of an abundance of caution, the operator will typically place the endotracheal tube around the insertion cord before inserting the insertion cord into the patient. If no endotracheal tube is needed, the precautionary endotracheal tube will be discarded after the operation without being used. Alternatively, the operator may determine that an endotracheal tube is needed during the operation and may pull the insertion cord out of the patient to place the endotracheal tube around the operating tip of the insertion cord, and then place the insertion cord back into the patient. However, multiple insertions of the insertion cord prolongs the operation and may cause additional irritation to the patient's trachea.

### BRIEF SUMMARY

The presently disclosed technology provides a system including an endoscope (e.g., a bronchoscope) with a detachable insertion cord so that an intubation may be performed during an endoscopy while maintaining the insertion cord in an endotracheal position. The system may, for instance, include a bronchoscope for intubating a patient, the bronchoscope comprising: a handle including: a first working channel connector; a first electrical connector; and a first mechanical pulley connector; and an insertion cord including: a working channel having a second working channel connector at an end of the working channel to form a first detachable connection with the first working channel connector; an electrical wire having a second electrical connector at an end of the electrical wire to form a second detachable connection with the first electrical connector; and a mechanical pulley cable having a second mechanical pulley connector at an end of the mechanical pulley cable to from a third detachable connection with the first mechanical pulley connector.

In some examples, the first detachable connection, the second detachable connection, and the third detachable connection are formed by mating a first half of an integrated connector with a second half of the integrated connector. The mechanical pulley cable may be a first mechanical pulley cable; the handle further includes a third mechanical pulley connector; and the insertion cord further includes a second mechanical pulley cable having a fourth mechanical pulley connector at an end of the second mechanical pulley cable to form a fourth detachable connection with the third mechanical pulley connector. The first mechanical pulley connector and the second mechanical pulley connector may have a first color; and the third mechanical pulley connector and the fourth mechanical pulley connector have a second color that is different than the first color. The first mechanical pulley connector includes a first magnet and the second mechanical pulley connector includes a second magnet to have an attractive force with the first magnet. The first working channel connector, the first electrical connector, and the first mechanical pulley connector may be formed into a first half of an integrated connector, and the second working channel connector, the second electrical connector, and the second mechanical pulley connector are formed into a second half of the integrated connector. The first working channel connector may extend through a center of the first half of the integrated connector; the first mechanical pulley connector may extend through the first half of the integrated connector spaced a distance from the center and extending parallel to the first working channel connector; and the first electrical connector may be a row of electrical contacts on a face of the first half of the integrated connector.

In some examples, the first half of the integrated connector includes a protuberance extending from a side of the first half of the integrated connector; and the second half of the integrated connector includes a sliding channel for receiving the protuberance. The sliding channel may be L-shaped such that the second half of the integrated connector is attached to the first half of the integrated connector with a linear engagement action and a rotational engagement action. In some instances, one of the first working channel connector or the second working channel connector is a first friction fit connector; and one of the first working channel connector or the second working channel connector is a second friction fit connector for sliding into the first friction fit connector with a linear engagement action. The first electrical connector may include a mount with an indented channel and a plurality of conductive pads disposed in the indented channel; and the second electrical connector may include a wire end with a plurality of outwardly facing conductive contacts for mating with the plurality of conductive pads when the wire end is placed in the indented channel. The first electrical connector may include a first row of electrical contacts on a first face of the first half of the integrated connector, and the second electrical connector may include a second row of electrical contacts that aligns with the first row of electrical contacts when the first half of the integrated connector is mated with the second half of the integrated connector. The first row of electrical contacts may include a plurality of metal bumps and the second row of electrical contacts may include a plurality of metal recesses to receive the plurality of metal bumps. The handle may include an access door movable between an open position and a closed position; and the first working channel connector, the first mechanical pulley connector, and the first electrical connector are exposed when the access door is in the open position.

In some examples, a system for performing an endoscopy and an intubation on a patient, the system comprising: a first working channel connector at a housing; a first electrical connector at the housing; and one or more first mechanical pulley connectors at the housing; a working channel disposed in an insertion cord and having a second working channel connector at an end of the working channel to form a first detachable connection with the first working channel connector; an electrical wire disposed in the insertion cord having a second electrical connector at an end of the electrical wire to form a second detachable connection with the first electrical connector; and a mechanical pulley cable disposed in the insertion cord having one or more second mechanical pulley connectors at an end of the mechanical pulley cable to from a third detachable connection with the one or more first mechanical pulley connectors. In some examples, the housing is a handle of a bronchoscope.

In some examples, a device for performing a bronchoscopy on a patient comprises: a housing including: a first working channel connector; a first electrical connector; and a first mechanical pulley connector; and an insertion cord including: a second working channel connector at a detachable end of the insertion cord to form a first detachable connection with the first working channel connector; a second electrical connector at the detachable end of the insertion cord to form a second detachable connection with the first electrical connector; and a second mechanical pulley connector at the detachable end of the insertion cord to form a third detachable connection with the first mechanical pulley connector.

In some examples, engaging a first half of an integrated connector with a second half of the integrated connector causes: the first working channel connector to mate with the second working channel connector; the first electrical connector to mate with the second electrical connector; and the first mechanical pulley connector to mate with the second mechanical pulley connector. The first mechanical pulley connector may include a recess and the second mechanical pulley connector may include a disc to be received by the recess when the first half of the integrated connector is rotatably engaged with the second half of the integrated connector. The first detachable connection, the second detachable connection, and the third detachable connection can be made with a single engagement action.

The foregoing is intended to be illustrative and is not meant in a limiting sense. Many features of the embodiments may be employed with or without reference to other features of any of the embodiments. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of apparatuses, systems, and methods consistent with the disclosed subject matter and, together with the description, serves to explain advantages and principles consistent with the disclosed subject matter, in which:

FIG. 1A illustrates an example system including an endoscope with a detachable insertion cord and an access door in a closed position which may form at least a portion of any of the systems discussed herein;

FIG. 1B illustrates an example system including an endoscope with a detachable insertion cord and an access door in an open position which may form at least a portion of any of the systems discussed herein;

FIG. 2A illustrates an example of electrical connector which may form at least a portion of any of the systems discussed herein;

FIG. 2B illustrates an example of electrical connector which may form at least a portion of any of the systems discussed herein;

FIG. 2C illustrates an example detachable connection formed by a first electrical connector and a second electrical connector which may form at least a portion of any of the systems discussed herein;

FIG. 3A illustrates an example conductive pad of an electrical connector which may form at least a portion of any of the systems discussed herein;

FIG. 3B illustrates an example conductive contact of an electrical connector which may form at least a portion of any of the systems discussed herein;

FIG. 4A illustrates an example detachable insertion cord disconnected from a handle of an endoscope which may form at least a portion of any of the systems discussed herein;

FIG. 4B illustrates an example detachable insertion cord forming a detachable connection for mechanical pulley cables of an endoscope which may form at least a portion of any of the systems discussed herein;

FIG. 5A illustrates an example of a first mechanical pulley connector disconnected from a second mechanical pulley connector which may form at least a portion of any of the systems discussed herein;

FIG. 5B illustrates an example of a first mechanical pulley connector forming a detachable connection with a second mechanical pulley connector which may form at least a portion of any of the systems discussed herein;

FIG. 6A illustrates an example of a first working channel connector disconnected from a second working channel connector which may form at least a portion of any of the systems discussed herein;

FIG. 6B illustrates an example of a first working channel connector forming a detachable connection with a second working channel connector which may form at least a portion of any of the systems discussed herein;

FIG. 6C illustrates an example of a first working channel connector disconnected from a second working channel connector which may form at least a portion of any of the systems discussed herein;

FIGS. 7A-7C illustrate an example endoscope with a housing, a detachable insertion cord and an access door in an open position which may form at least a portion of any of the systems discussed herein;

FIGS. 8A and 8B illustrate an example integrated connector which may form at least a portion of any of the systems discussed herein;

FIG. 9A illustrates an example of at least a portion of a first half of an integrated connector which may form at least a portion of any of the systems discussed herein;

FIG. 9B illustrates an example of at least a portion of a second half of an integrated connector which may form at least a portion of any of the systems discussed herein;

FIG. 10A illustrates an example first mechanical pulley connector which may form at least a portion of any of the systems discussed herein;

FIG. 10B illustrates an example second mechanical pulley connector which may form at least a portion of any of the systems discussed herein;

FIG. 11A illustrates an example electrical connector which may form at least a portion of any of the systems discussed herein;

FIG. 11B illustrates an example bump portion of an electrical connector which may form at least a portion of any of the systems discussed herein;

FIG. 11C illustrates an example electrical connector which may form at least a portion of any of the systems discussed herein;

FIG. 11D illustrates an example receiver portion of an electrical connector which may form at least a portion of any of the systems discussed herein; and

FIG. 12 illustrates example operations of a method for performing an endoscopy and/or an intubation which may be performed by any of the systems discussed herein.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the presently disclosed technology may include a variety of combinations and/or integrations of the embodiments described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### II. General Architecture

In some instances, the system disclosed herein allows an operator to insert an insertion cord of an endoscope, such as a bronchoscope, into a patient to inspect the patient's airways. In examples where intubation is necessary, the insertion cord can be detached from a handle of the bronchoscope to enable passing an endotracheal tube over the detachable end of the insertion cord. The bronchoscope makes three detachable connections to the insertion cord: mechanical connections with mechanical connectors which connect mechanical pulley wires in a pulley system that cause the tip of the bronchoscope to flex; connections for the working channel with working channel connectors which allows suction of liquids through the bronchoscope as well as injection of medication into the airways; and electrical connections with electrical connectors which provides power to a camera and a light at the operating tip of the bronchoscope to facilitate airway inspection with a computer screen. These three separate connections form one or more detachable connections at the handle of the bronchoscope. The connections provide an ease of connection and disconnection by operators, for instance, during a procedure. Due to size limitations of human airways, the connectors may fit inside a 9 mm diameter endotracheal tube.

In some scenarios the bronchoscope can have multiple insertion cords that can be interchangeable with the handle. For instance, the handle can be usable with multiple insertion cords having different calibers. Accordingly, a single bronchoscope can be used for different applications and/or with patients of different sizes, reducing equipment costs for the bronchoscopist.

As such, the systems disclosed herein may provide more efficient techniques for performing bronchoscopies by reducing the need to place an endotracheal tube over the operating tip before insertion (which sometimes results in wasted endotracheal tubes) and avoiding the need to extract the insertion cord from the patient during placement of the endotracheal tube. Moreover, the particular arrangements of connectors disclosed herein may provide for simple and quick detachments and reattachments of the insertion cord to a housing of the bronchoscope with few engagement actions (e.g., less than three) so that the operator may detach and reattach the insertion cord without assistance and/or with one hand during the operation.

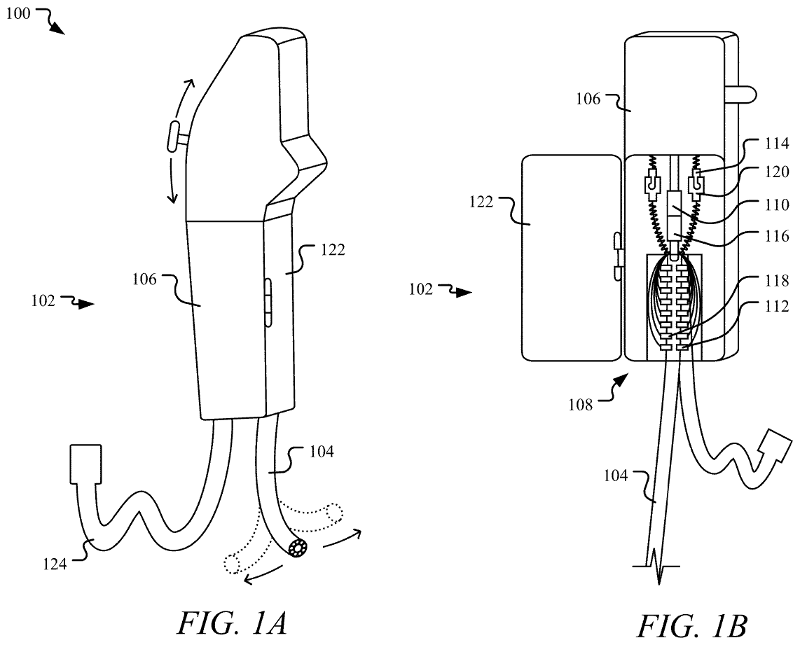

FIGS. 1A and 1B illustrate a system 100 including an endoscope, such as a bronchoscope 102, with a detachable insertion cord 104 for performing an endoscopy (e.g., a bronchoscopy) and/or an intubation during the endoscopy by detaching the insertion cord 104. The bronchoscope 102 may include a handle with a housing 106 for containing various connectors for connecting and disconnecting the insertion cord 104 from the housing 106. The housing may be formed of a rigid, durable material not subject to damage by bodily fluids (e.g., plastic, metal, carbon fiber, etc.) and has an end 108 to receive the insertion cord 104. A first working channel connector 110, a first electrical connector 112, and a first mechanical pulley connector 114 may be contained within the housing 106. The insertion cord 104 may include a second working channel connector 116, a second electrical connector 118, and a second mechanical pulley connector 120. The housing 106 may include an access door 122 that is movable between an open position (e.g., FIG. 1B) and a closed position (e.g., FIG. 1A), such that the first working channel connector 110, the first electrical connector 112, and the first mechanical pulley connector 114 are exposed when the access door 122 is in the open position. The access door 122 may be hinged or slide along rails to move between the open position and the closed position. Moreover, when the insertion cord 104 is attached to the handle, the second working channel connector 116, the second electrical connector 118, and the second mechanical pulley connector 120 (e.g., attached to the detachable end of insertion cord 104) may be exposed when the access door 122 is in the open position. As illustrated in FIGS. 1A and 1B, the various connectors in the housing 106 and at the end of the insertion cord 104 may be separate and distinct connectors forming detachable connections with separate engagement actions. However, in some examples, the connectors in the housing 106 may be formed into a first half of an integrated connector (e.g., the integrated connector 800 discussed below) and the connectors at the end of the insertion cord 104 may be formed into a second half of the integrated connector so that one or more engagement actions on a single connector cause all three detachable connections to form (e.g., the first detachable connection between the first working channel connector 110 and the second working channel connector 116, the second detachable connection between the first electrical connector 112 and the second electrical connector 118, and/or the third detachable connection between the first mechanical pulley connector 114 and the second mechanical pulley connector 120). In some instances, the one or more engagement actions may be exclusive engagement actions in that the features of the integrated connector guide proper alignment of the connectors and prevents alternative engagement actions that fail to properly align the three connections. Although a bronchoscope is discussed herein, it is to be understood that the endoscope may include other types of endoscopes, such as a cystoscope, a nephroscope, an arthroscope, a colonoscope, or a laparoscope.

In some examples, the insertion cord 104 may include multiple components bundled and/or sheathed together. For instance, the insertion cord 104 may include a working channel, an electrical wire and one or more mechanical pulley cables. The insertion cord 104 can be 9 mm in diameter or less and may be able to fit into at least an endotracheal tube with an inner diameter of 9 mm. In some instances, the insertion cord 104 is attachable, detachable, and re-attachable quickly and simply with one or more engagement actions (e.g., a single engagement action, two engagement actions, three engagement actions, etc.) such as a linear engagement action (e.g., a push), and/or a rotational engagement action (e.g., a twist). As such, the insertion cord 104 may be detachable by a single operator, in some instances, with one hand, during an operation so that an endotracheal tube may be slid over the detached end of the insertion cord 104, and the insertion cord 104 can be re-attached with the endotracheal tube in place ready to be positioned in the patient's trachea. The insertion cord 104 may remain in place inside the patient (e.g., in the bronchial and tracheal regions) during the detachment of the insertion cord 104, placement of the endotracheal tube over the insertion cord 104, and re-attachment of the insertion cord 104, resulting in less total intubations, less irritation of the patient's trachea, and a more efficient technique in terms of time and supplies because, in some instances, less tracheal tubes will be unnecessarily placed over the insertion cord 104 prior to insertion into the patient.

In some examples, the insertion cord 104 includes the working channel which may be a flexible tube for transporting fluids between the patient and the endoscope (e.g., the handle housing 106 of the bronchoscope 102). For instance, the working channel may collect lung tissue sample from the patient, send radiographic media into the patient, perform laser therapy, remove a foreign object, perform suction (e.g., for microbiological culturing), and/or insert catheters. The insertion cord 104 may include the electrical wire may be bundle of smaller wires separated by insulator within the bundle. For instance, the electrical wire which may include one or more wires connecting a camera at an operating end of the insertion cord 104 to devices (e.g., batteries, monitors, printed circuit boards (PCB)s, etc.) outside the patient and/or inside the endoscope (e.g., the housing 106 of the bronchoscope 102). The electrical wire may include one or more wires connecting a light at the operating end of the insertion cord 104 to a power source. In some instances, the electrical wire may include 8 wires that connect any electrical components operating in the insertion cord 104 to a PCB (e.g., having 8 electrical contacts) in the housing 106. The insertion cord 104 may include one or more mechanical pulley cables to steer or deflect the operating end or tip of the insertion cord 104. For instance, the one or more mechanical pulley cables may be flexible or rigid cables (e.g., plastic, metal, nylon, etc.) that attach the tip of the insertion cord 104 at the operating end to a steering knob so that movements of the steering knob (e.g., a sliding motion or a twisting motion) cause the operating end to move or deflect in lateral directions (e.g., right or left) and/or in linear directions (e.g., up or down). In some instances, the one or more mechanical pulley cables may include a first mechanical pulley for pulling the operating end in a first direction (e.g., right) and a second mechanical pulley for pulling the operating end in a second direction (e.g., left). The bronchoscope 102 may further include a video adapter 124 extending from the housing 106 for connecting the bronchoscope 102 to a monitor or display device.

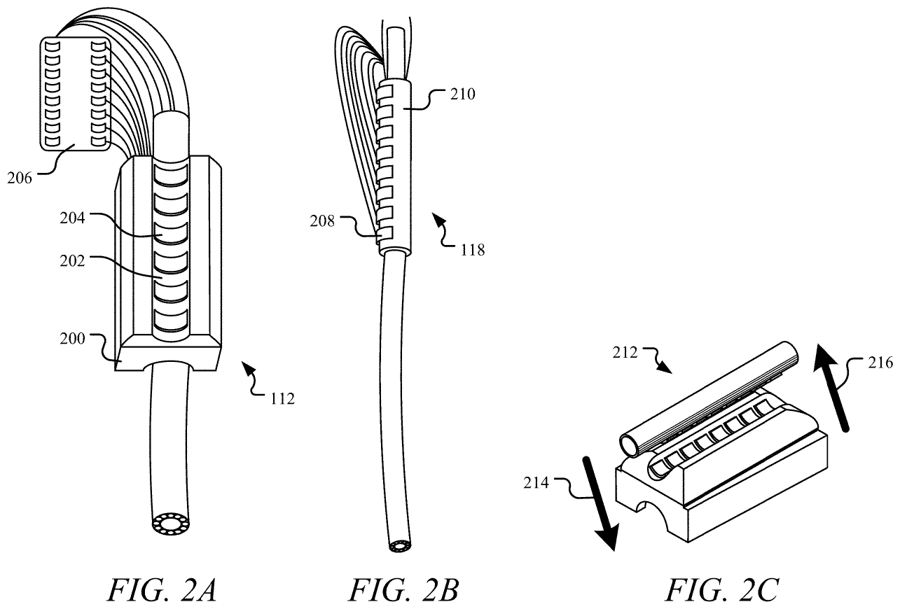

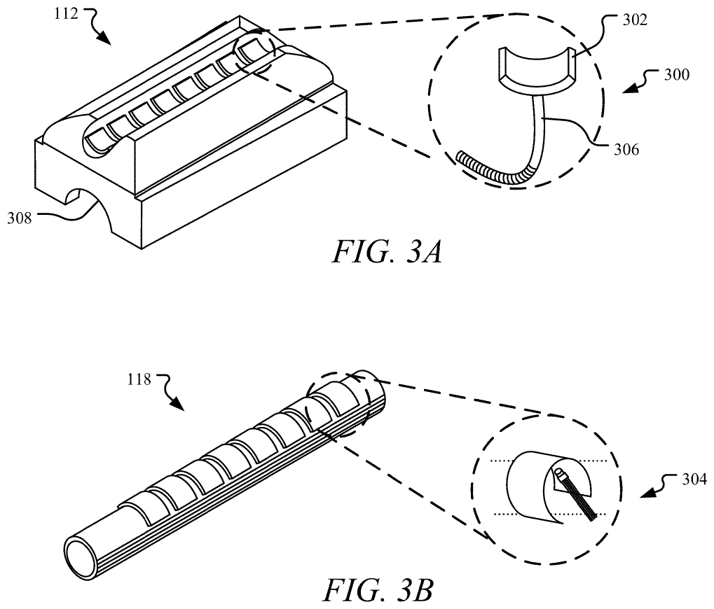

FIGS. 2A-3B illustrate examples of the first electrical connector 112 and the second electrical connector 118 which may form at least a portion of any of the systems discussed herein. FIG. 2A illustrates the first electrical connector 112 with a mount 200 to couple the first electrical connector 112 to an inner surface of the housing 106. The mount 200 may be a block (e.g., having a substantially rectangular footprint) and may have an indented channel 202 on a top surface with one or more conductive pad(s) 204 disposed in the indented channel 202 and spaced apart. The indented channel 202 may have a profile (e.g., a curved profile) for receiving the second electrical connector 118 which may have a shape (e.g., a round shape such as a cylindrical shape) to mate with the profile of the indented channel 202. The one or more conductive pad(s) 204 may couple the first electrical connector 112 to a PCB 206 in the housing 106. For instance, the one or more conductive pad(s) 204 may include 8 conductive pads 204 that correspond to 8 input pins of the PCB 206.

In some examples, the second electrical connector 118 may include a wire end at the detachable end of the insertion cord 104. The wire end may be a termination point for the electrical wire (e.g., a plurality of electrical wires), which may terminate at one or more conductive contact(s) 208 facing outwardly from the detachable end of the insertion cord 104. The one or more conductive contact(s) 208 may have a one-to-one correspondence with the one or more conductive pad(s) 204 of the first electrical connector 112. The second electrical connector 118 may include a cylindrical sheath 210 over the detachable end of the insertion cord 104 for positioning the one or more conductive contact(s) 208 in place to form the second electrical connector 118. In some instances, a detachable connection 212 may be formed by moving the second electrical connector 118 towards the first electrical connector 112 with a first lateral engagement action 214 (e.g., “lateral” meaning in a direction perpendicular to the insertion cord 104 and/or perpendicular to a y-axis defined by a direction the insertion cord 104 is inserted into the housing 106) until the second electrical connector 118 mates with the first electrical connector 112. In the detachable connection 212, the one or more conductive contact(s) 208 may contact and form electrical connections with the one or more conductive pad(s) 204 to transmit electricity between the handle of the bronchoscope 102 and electrical components disposed in or on the insertion cord 104. A second lateral engagement action 216 in an opposite direction as the first lateral engagement action 214 may pull the second electrical connector 118 apart from the first electrical connector 112, breaking the electrical connections between the one or more conductive contact(s) 208 and the one or more conductive pad(s) 204 (e.g., when the insertion cord 104 is being detached from the handle).

Turning now to FIGS. 3A and 3B, an electrical pad 300 of the one or more electrical pad(s) 204 forming the first electrical connector 112 may include a metal receiver 302 which may be concavely curved or otherwise shaped to mate with an electrical contact 304 of the one or more electrical contact(s) 208. The metal receiver 302 may be disposed in the indented channel 202 with a metal stem 306 extending from the metal receiver 302, through the mount 200 and into a wire passageway 308 disposed under the mount 200. An end of the metal stem 306 may couple to a lead line (e.g., via a solder) which connects the metal receiver 302 to a particular contact of the PCB 206. The electrical contact 304 may be convexly curved and may be attached (e.g., with adhesive) to an outer surface of the cylindrical sheath 210 wrapping around the insertion cord 104 with an end of the electrical wire passing from inside the insertion cord 104 and the cylindrical sheath 210 to an inner surface of the electrical contact 304 (e.g., via a solder). As such, the electrical contact 304 may be electrically connected to any electrical components disposed in or on the insertion cord 104.

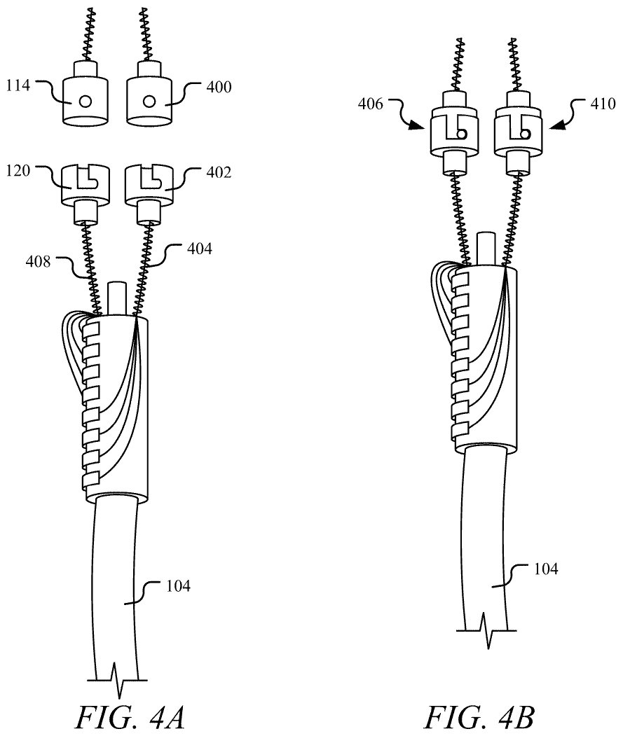

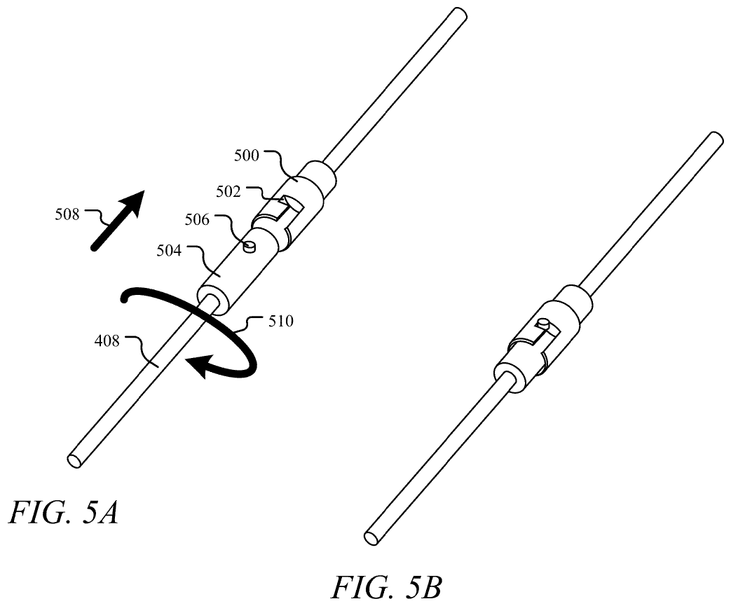

FIGS. 4A-5B illustrate examples of the first mechanical pulley connector 114, the second mechanical pulley connector 120, the second electrical connector 118 and the second working channel connector 116 which may form at least a portion of any of the systems discussed herein. FIG. 4A illustrates the first mechanical pulley connector 114 and the second mechanical pulley connector in a disengaged or separated position. FIG. 4B illustrates the first mechanical pulley connector 114 and the second mechanical pulley connector in an engaged or connected position. FIGS. 4A and 4B also illustrate a third mechanical pulley connector 400 disposed in the housing 106 to mate with a fourth mechanical pulley connector 402 disposed on an end of a second mechanical pulley cable 404. For instance, the first mechanical pulley connector 114 and the second mechanical pulley connector 120 may mate together forming a first detachable connection 406 for a first mechanical pulley cable 408 that steers the operating tip of the insertion cord 104 in a first direction. The third mechanical pulley connector 400 and the fourth mechanical pulley connector 402 may mate together forming a second detachable connection 410 for the second mechanical pulley cable 404 that steers the operating tip of the insertion cord 104 in a second direction. The second mechanical pulley connector 120 and/or the fourth mechanical pulley connector 402 may extend from the detachable end of the insertion cord 104 by extending out from the cylindrical sheath 210 of the second electrical connector 118 beyond where the detachable end of the insertion cord 104 terminates. The first mechanical pulley connector 114 and/or the third mechanical pulley connector 400 may be disposed in the housing 106 of the handle and may be accessible when the access door 122 is in the open position.

Turning now to FIGS. 5A and 5B, the first mechanical pulley connector 114 and/or the third mechanical pulley connector 400 may include a first substantially cylindrical body 500 with a sliding channel 502 in a side surface of the first substantially cylindrical body 500. In some instances, the sliding channel 502 may be L-shaped in that it may have a first linear channel in a first direction connected to a second linear channel in a second direction perpendicular to the first direction. The second mechanical pulley connector 120 and/or the fourth mechanical pulley connector 402 may include a second substantially cylindrical body 504 for inserting into an opening of the first substantially cylindrical body 500 to form the first detachable connection 406 and/or the second detachable connection 410. Moreover, in forming the first detachable connection 406 and/or the second detachable connection 410, a protuberance 506 extending perpendicularly from the second substantially cylindrical body 504 may slide into the sliding channel 502. For instance, forming the first detachable connection 406 and/or the second detachable connection 410 may include a first engagement action 508 in a linear direction and a second engagement action 510 in a rotational direction such that the protuberance 506 slides into and along the sliding channel 502 until the second mechanical pulley connector 120 and/or the fourth mechanical pulley connector 402 are locked in place with respect to the first mechanical pulley connector 114 and/or the third mechanical pulley connector 400. In some instances, the first mechanical pulley connector 114 and/or the third mechanical pulley connector 400 may include a first magnet and the second mechanical pulley connector 120 and/or the fourth mechanical pulley connector 402 may include a second magnet for creating an attractive force with the first magnet, which may assist in aligning the mechanical pulley connectors together and providing a force to create the first detachable connection 406 and/or the second detachable connection 410. In some examples, the first mechanical pulley connector 114 and the second mechanical pulley connector 120 may have a first color to color code the first mechanical pulley connector 114 and the second mechanical pulley connector 120 with a first visual indicator showing that the first mechanical pulley connector 114 is meant to couple with the second mechanical pulley connector 120. The third mechanical pulley connector 400 and the fourth mechanical pulley connector 402 may have a second color that is different than the first color to color code the third mechanical pulley connector 400 and the fourth mechanical pulley connector 402 with a second visual indicator showing that the third mechanical pulley connector 400 is meant to couple with the fourth mechanical pulley connector 402.

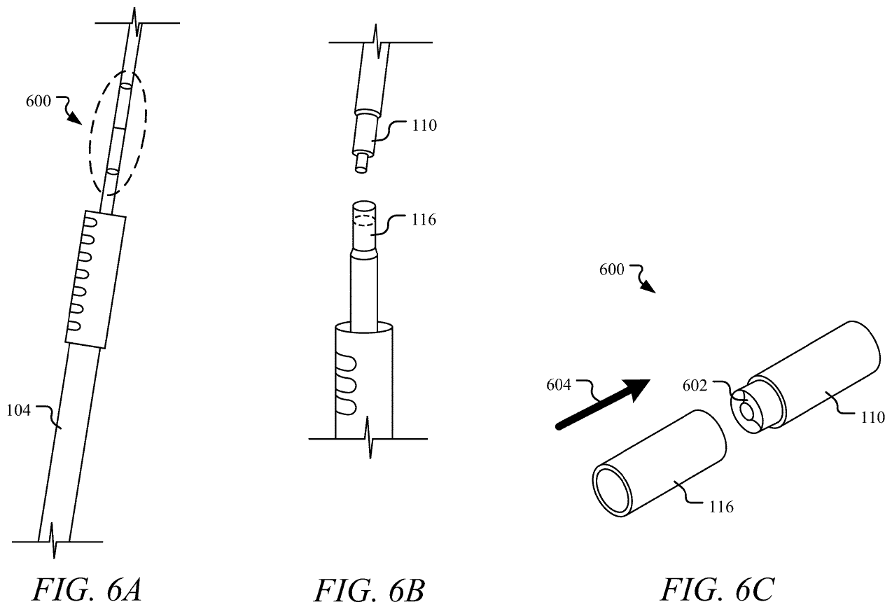

FIGS. 6A-6C illustrate examples of the first working channel connector 110 and the second working channel connector 116 which may form at least a portion of any of the systems discussed herein. FIG. 6A illustrates the first working channel connector 110 and the second working channel connector 116 in a disengaged or separated position in which the working channel in the insertion cord 104 is detached from the handle of the bronchoscope 102. FIG. 6B illustrates the first working channel connector 110 and the second working channel connector 116 forming a detachable connection 600 for the working channel in the insertion cord 104 and the handle of the bronchoscope 102. FIG. 6C illustrates the first working channel connector 110 and the second working channel connector 116 in a disengaged or separated position.

In some examples, the second working channel connector 116 may extend from the detachable end of the insertion cord 104 and/or out an end of the cylindrical sheath 210 (e.g., along a central axis of the cylindrical sheath 210). The first working channel connector 110 may be a first cylindrical tube and the second working channel connector 116 may be a second cylindrical tube for receiving the first cylindrical tube. The first cylindrical tube and/or the second cylindrical tube may be formed from a flexible material, such as rubber, plastic, nylon, etc., so that the detachable connection 600 is formed via a friction fitting between the first working channel connector 110 and the second working channel connector 116. The first working channel connector 110 may include an inner recessed tip 602 surrounded by an inner circular indentation which may receive an 0-ring to provide sealing for the detachable connection 600 to prevent fluid leakage. The detachable connection 600 of the first working channel connector 110 with the second working channel connector 116 may be formed with a single, linear engagement action 604. The first working channel connector 110 and/or the second working channel connector 116 may be friction-fit connectors for forming a friction-fit connection. In some instances, upon attaching the first working channel connector 110 with the second working channel 116, a continuous working channel is formed. This working channel can be used to delivery various fluids and/or substances into the patient, such as endobronchial blockers to control pulmonary bleeding.

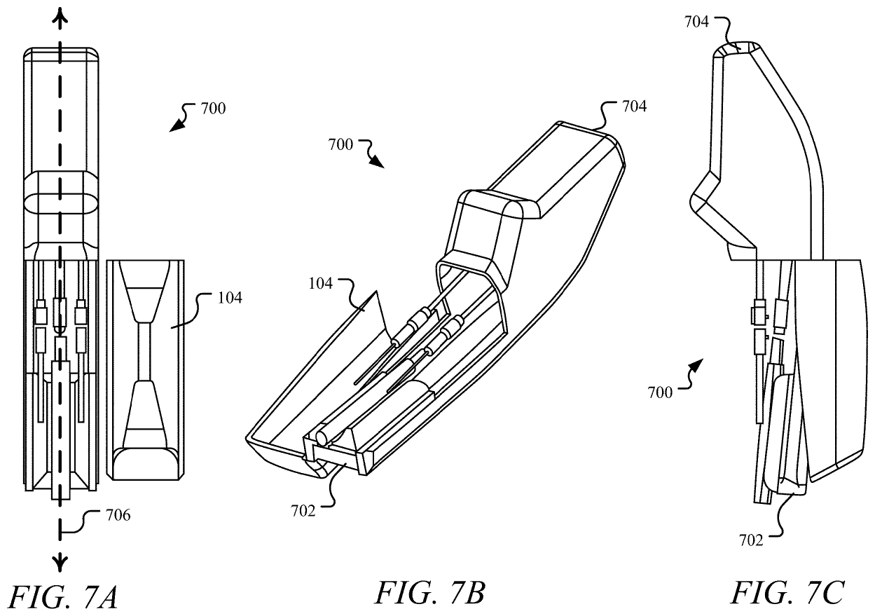

FIGS. 7A-7C illustrate an example endoscope 700 such as the bronchoscope 102 including the handle with the housing 106 with the access door 122 in the opened position which may form at least a portion of any of the systems discussed herein. The housing 106 may include a first end 702 for receiving the detachable end of the insertion cord 104 and a second end 704 opposite the first end 702. A vertical or y-axis 706 of the housing 106 may be defined as extending between the first end 702 and the second end 704. In some instances, a bottom half of the housing 106 proximate to the first end 702 may be accessible via the access door 122 and may include the first working channel connector 110, the first electrical connector 112, and the first mechanical pulley connector 114. A top half of the housing 106 proximate to the second end 704 may include additional components of the bronchoscope 102, such as the PCB 206 a working channel apparatus (e.g., a fluid injector, a pump, a vacuum, etc.), and/or mechanical components for connecting the one or more mechanical pulley cables to the steering knob. In some examples (as discussed in greater detail below), the first working channel connector 110, the first electrical connector 112, and the first mechanical pulley connector 114 may be disposed outside the housing 106, such as below the first end 702.

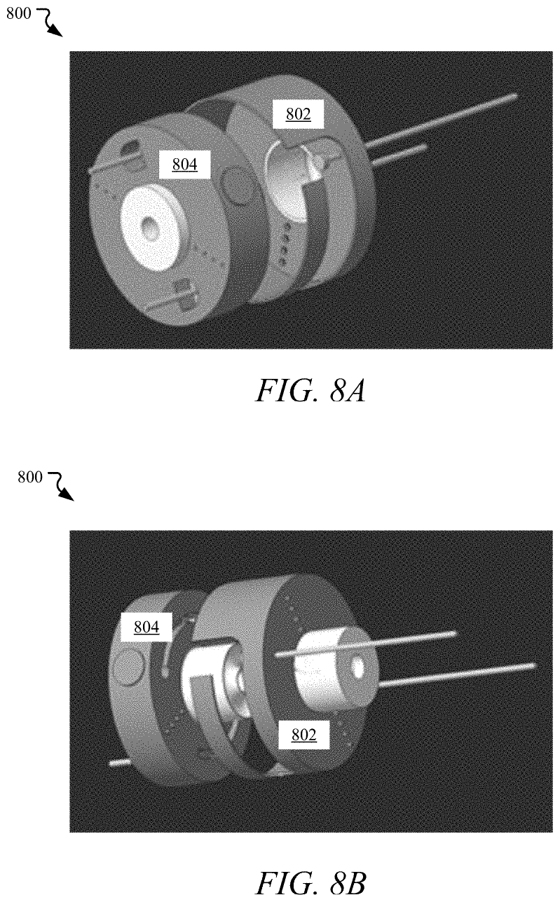

FIGS. 8A-10D illustrate examples of an integrated connector 800 with a first half 802 and a second half 804 for making a first detachable connection (e.g., the detachable connection 212 between the first electrical connector 112 and the second electrical connector 118), a second detachable connection (e.g., the first detachable connection 406 between the first mechanical pulley connector 114 and the second mechanical pulley connector 120 and/or the second detachable connection 410 between the third mechanical pulley connector 400 and the fourth mechanical pulley connector 402) , and a third detachable connection (e.g., the detachable connection 600 between the first working channel connector 110 and the second working channel connector 116), which may form at least a portion of any of the systems discussed herein.

In some instances, the first half 802 of the integrated connector 800 may be formed with the first working channel connector 110, the first electrical connector 112, and/or the first mechanical pulley connector 114, and the second half 804 may be formed with the second working channel connector 116, the second electrical connector 118 and/or the second mechanical pulley connector 120. As such, one or more engagement actions on a single connector—the integrated connector 800—may form the first detachable connection between the first working channel connector 110 and the second working channel connector 116, the second detachable connection between the first electrical connector 112 and the second electrical connector 118, and the third detachable connection between the first mechanical pulley connector 114 and the second mechanical pulley connector 120. The one or more engagement actions may include a linear engagement action (e.g., a pushing motion) and/or a rotational engagement action (e.g., a twisting motion) to move the first half 802 in an L-motion with respect to the second half 804, mating the first half 802 with the second half 804 to form a detachable connection of the integrated connector 800. In some instances, the integrated connector 800 may be disposed outside the housing 106, such as between 1 and 100 mm (e.g., 20 mm, 30 mm, 40 mm, 50 mm, etc.) below the handle

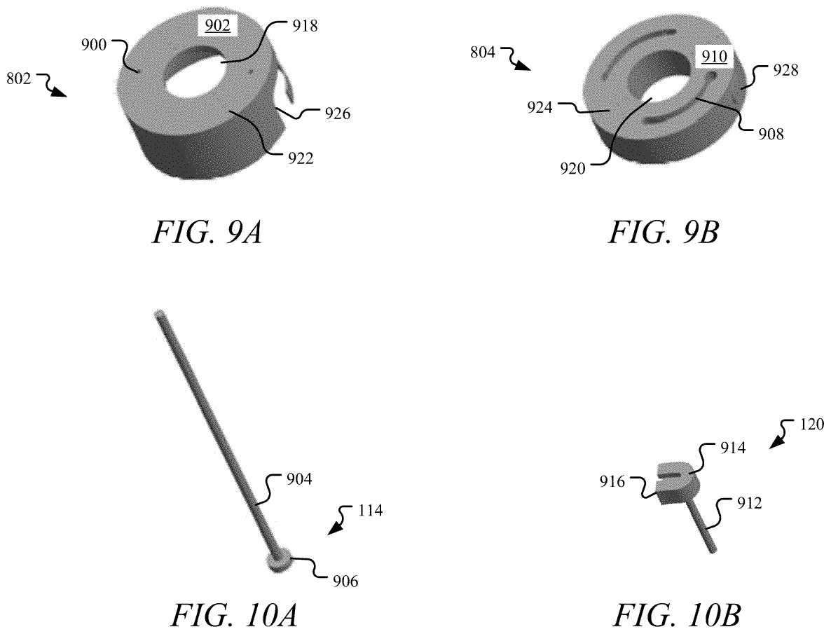

FIGS. 9A-10B illustrate the first half 802 of the integrated connector 800, the second half 804 of the integrated connector 800, first mechanical pulley connector 114, and the second mechanical pulley connector 120 which may form at least a part of any of the systems discussed herein. For instance, the first half 802 of the integrated connector 800 may include one or more cable opening(s) 900 on a first face 902 of the first half 802. The first face 902 may be substantially circular and the one or more cable opening(s) 900 may be located on the first face 902 spaced a distance apart from a center point of the first face 902. The one or more cable opening(s) 900 may extend from the first face 902, through the first half 802, and to an inner face underneath the first face 902. A first portion 904 of the mechanical pulley cable with the first mechanical pulley connector 114 may pass through one of the cable opening(s) 900 and a disc 906 of the first mechanical pulley connector 114 may be disposed in the first half 802 at the inner face opposite the first face 902. The second half 804 may include one or more cable opening channel(s) 908 on a second face 910 of the second half 804. The cable opening channel(s) 908 may be a slot, cut, or groove in the second half 804, extending from the second face 910, through the second half 804, and to an inner face underneath the second face 910. The cable opening channel(s) 910 may be located on the second face 910 spaced apart from the center point of the second face 910 such that the cable opening channel(s) 910 align with the cable opening(s) 900 on the first face 902 when the first half 802 of the integrated connector 800 is mated to the second half 804 of the integrated connector 800, for instance, with one or more engagement actions (e.g., a linear action, a rotational action, a traverse action, etc.). A second portion 912 of the mechanical pulley cable with the second mechanical pulley connector 120 may pass through the cable opening channel(s) 910 and a receiving portion 914 of the second mechanical pulley connector 120 may be disposed in the second half 804 at the inner face opposite the second face 910. A rotational engagement action to mate the first half 802 with the second half 804 may cause the second portion 912 of the mechanical pulley cable with the second mechanical pulley connector 120 to rotate, guided by the cable opening(s) 910, so that the disc 906 is guided into a recess 916 in a side of the receiving portion 914, forming the detachable connection 600 between the first mechanical pulley connector 114 and the second mechanical pulley connector 120. In some instances, the cable opening channel(s) 910 may extend circumferentially around approximately a sixth, quarter a third, or half of the second face 910 so that a rotational engagement action of about 60°, 90°, 120°, or 180° forms the detachable connection 600.

In some examples, the first face 902 of the first half 802 may include a first center opening 918 for receiving or holding the first working channel connector 110 and the second face 910 may include a second center opening 920 for receiving or holding the second working channel connector 116. The integrated connector 800 may align the first working channel connector 110 and the second working channel connector 116 by aligning the first center opening 918 and the second center opening 920 and the linear engagement action on the integrated connector 800 may form the detachable connection 600 between the first working channel connector 110 and the second working channel connector 116. Moreover, the first face 902 may include one or more first electrical connector openings 922 and the second face 910 may include one or more second electrical connector openings 924 to align with the first electrical connector openings 922 when the integrated connector 800 is detachably connected. In some examples, the one or more first electrical connectors openings 922 and the one or more second electrical connector openings 924 may be arranged in one or more rows, such as a first row on a first half of the first face 902 and a second row aligned with the first row on a second half of the first face 902. The first half 802 of the integrated connector 800 may have a sliding channel 926 on its side, such as an L-shaped sliding channel. The second half 804 may have a protuberance 928 extending from its side to slide into the sliding channel 926 to align and mate the first half 802 with the second half 804, for instance, with one or more engagement actions. In some instances, a linear engagement action pushing the first half 802 towards the second half 804 (e.g., until the inner face of the first half 802 contacts the inner face of the second half 804) may push the protuberance 928 into the sliding channel 926 to align and connect the first working channel connector 110 with the second working channel connector 116. A rotational engagement action (e.g., subsequent to the linear engagement action) rotating the second half 804 relative to the first half 802 until the connector openings spaced a distance apart from the center are aligned (e.g., the one or more cable opening(s) 900, the one or more first electrical connector openings 922, and/or the one or more second electrical connector openings 924). As such, the connectors may be formed into or integrated into the integrated connector 800 so that a single connector forms the detachable connection 212 between the first electrical connector 112 and the second electrical connector 118, the first detachable connection 406 between the first mechanical pulley connector 114 and the second mechanical pulley connector 120, the second detachable connection 410 between the third mechanical pulley connector 400 and the fourth mechanical pulley connector, and the detachable connection 600 between the first working channel connector 110 and the second working channel connector 116. Moreover, a reverse rotational disengagement action and a (e.g., subsequent) reverse linear action may cause the first half 802 and the second half 804 and the connectors of the integrated connector 800 to disengage, detaching the insertion cord 104 from the housing 106.

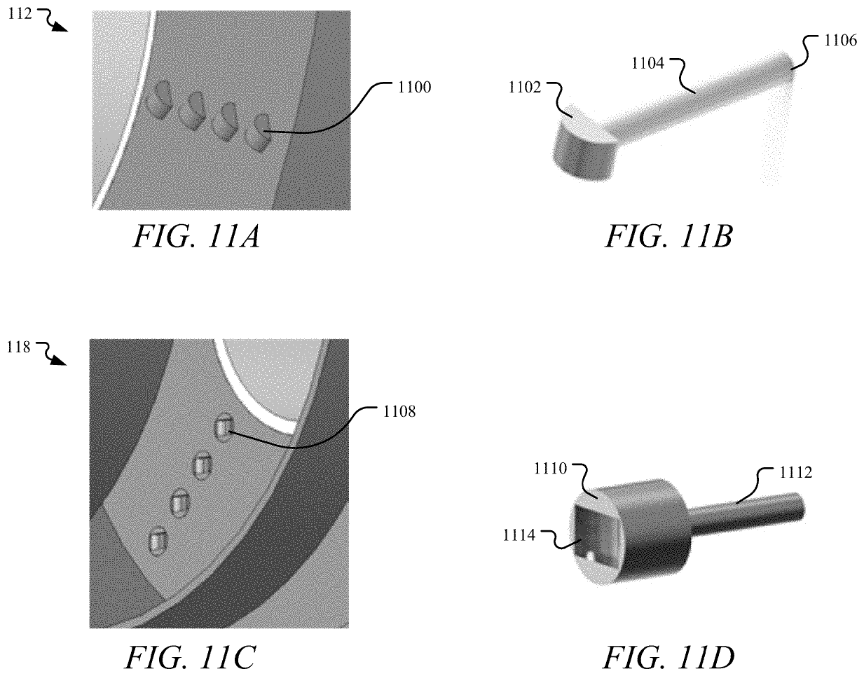

FIGS. 11A-11D illustrate the first electrical connector 112 and the second electrical connector 118 which may form at least a part of any systems discussed herein. The first electrical connector 112 may be one or more electrical contact(s) 1100 (e.g., arranged in one or more rows on the first face 902 of the first half 802). The electrical contact(s) 1100 may include a bump portion 1102 (e.g., a metal bump shaped as a hemi-circle, a crest, and/or a half cylinder) at an end of a stem 1104. The stem 1104 may pass through the first electrical connector opening 922 with the bump portion 1102 protruding from the inner face of the first half 802 and a distal end 1106 of the stem coupling to the electrical wire. The second electrical connector 118 may be one or more electrical contact receiver(s) 1108 for receiving and mating with the electrical contact(s) 1100. For instance, the electrical contact receiver(s) 1108 may include a receiver portion 1110 and a stem 1112. The receiver portion 1110 may include a recess 1114 with a same and/or complimentary shape as the bump portion 1102. The receiver portion 1110 may be cylindrical shaped or rectangular prism shaped and may be recessed into the inner face of the second half 804 of the integrated connector 800 (e.g., via a shelf cut into the inner face). The electrical contact(s) 1100 and the electrical contact receiver(s) 1108 may be formed of a highly conductive metal such as gold.

In some examples, with respect to being located in the housing 106 or in the detachable end of the insertion cord 104, the first working channel connector 110 may be reversible with the second working channel connector 116, the first electrical connector 112 may be reversible with the second electrical connector 118, and the first mechanical pulley connector 114 may be reversible with respect to the second mechanical pulley connector 120. Likewise, the first half 802 of the integrated connector 800 and/or any features of the first half 802 may be reversible with the second half 804 and/or any features of the second half 804 (e.g., may be disposed in the housing 106 or at the detachable end of the insertion cord 104).

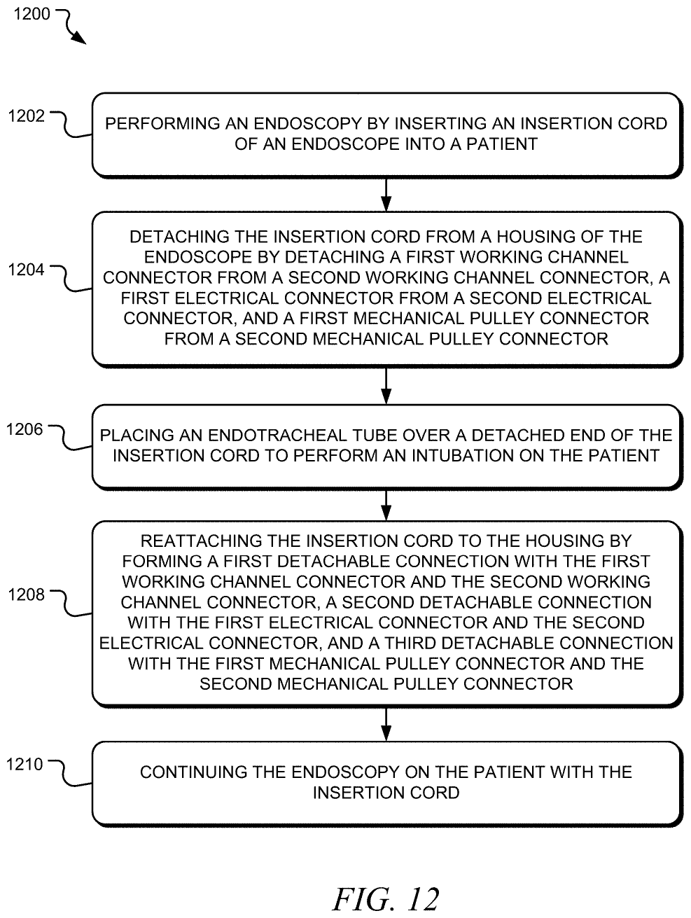

FIG. 12 illustrates an example method 1200, which may be performed by any of the systems discussed herein for performing an endoscopy (e.g., a bronchoscopy) and/or intubation for a patient. At step 1202, the method 1200 may include performing an endoscopy by inserting the insertion cord 104 of an endoscope (e.g., the bronchoscope 102) into a patient. At step 1204, the method 1200 may include detaching the insertion cord 104 from the housing 106 of the endoscope by detaching one or more of the first working channel connector 110 from the second working channel connector 116, the first electrical connector 112 from the second electrical connector 118, or the first mechanical pulley connector 114 from the second mechanical pulley connector 120. At step 1206, the method 1200 may include placing an endotracheal tube over a detached end of the insertion cord 104 to perform an intubation on the patient. At step 1208, the method 1200 may include reattaching the insertion cord 104 to the housing 106 of the endoscope by forming a first detachable connection with the first electrical connector 112 and the second electrical connector 118, a second detachable connection with the first electrical connector 112 and the second electrical connector 118, and a third detachable connection with the first mechanical pulley connector 114 and the second mechanical pulley connector 120. At step 1210, the method 1200 may include continuing the endoscopy on the patient with the insertion cord 104.

It is to be understood that the specific order or hierarchy of steps in the method depicted in FIG. 12 and other methods disclosed herein are instances of example approaches and can be rearranged while remaining within the disclosed subject matter. For instance, any of the steps depicted in FIG. 12 may be omitted, repeated, performed in parallel, performed in a different order, and/or combined with any other of the steps depicted in FIG. 12.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A bronchoscope for intubating a patient, the bronchoscope comprising:
a handle including:
a first working channel connector;
a first electrical connector; and
a first mechanical pulley connector; and
an insertion cord including:
a working channel having a second working channel connector at an end of the working channel to form a first detachable connection with the first working channel connector;
an electrical wire having a second electrical connector at an end of the electrical wire to form a second detachable connection with the first electrical connector; and
a mechanical pulley cable having a second mechanical pulley connector at an end of the mechanical pulley cable to from a third detachable connection with the first mechanical pulley connector.

2. The bronchoscope of claim 1, wherein the first detachable connection, the second detachable connection, and the third detachable connection are formed by mating a first half of an integrated connector with a second half of the integrated connector.

3. The bronchoscope of claim 1, wherein:
the mechanical pulley cable is a first mechanical pulley cable;
the handle further includes a third mechanical pulley connector; and
the insertion cord further includes a second mechanical pulley cable and a fourth mechanical pulley connector at an end of the second mechanical pulley cable to form a fourth detachable connection with the third mechanical pulley connector.

4. The bronchoscope of claim 3, wherein:
the first mechanical pulley connector and the second mechanical pulley connector have a first color; and
the third mechanical pulley connector and the fourth mechanical pulley connector have a second color that is different than the first color.

5. The bronchoscope of claim 1, wherein the first mechanical pulley connector includes a first magnet and the second mechanical pulley connector includes a second magnet to have an attractive force with the first magnet.

6. The bronchoscope of claim 1, wherein the first working channel connector, the first electrical connector, and the first mechanical pulley connector are formed into a first half of an integrated connector, and the second working channel connector, the second electrical connector, and the second mechanical pulley connector are formed into a second half of the integrated connector.

7. The bronchoscope of claim 6, wherein:
the first working channel connector extends through a center of the first half of the integrated connector;
the first mechanical pulley connector extends through the first half of the integrated connector spaced a distance from the center and extending parallel to the first working channel connector; and
the first electrical connector is a row of electrical contacts on a face of the first half of the integrated connector.

8. The bronchoscope of claim 6, wherein:
the first half of the integrated connector includes a protuberance extending from a side of the first half of the integrated connector; and
the second half of the integrated connector includes a sliding channel for receiving the protuberance.

9. The bronchoscope of claim 8, wherein the sliding channel is L-shaped such that the second half of the integrated connector is attached to the first half of the integrated connector with a linear engagement action and a rotational engagement action.

10. The bronchoscope of claim 1, wherein:
one of the first working channel connector or the second working channel connector is a first friction fit connector; and
one of the first working channel connector or the second working channel connector is a second friction fit connector for sliding into the first friction fit connector with a linear engagement action.

11. The bronchoscope of claim 1, wherein:
the first electrical connector includes a mount with an indented channel and a plurality of conductive pads disposed in the indented channel; and
the second electrical connector includes a wire end with a plurality of outwardly facing conductive contacts for mating with the plurality of conductive pads when the wire end is placed in the indented channel.

12. The bronchoscope of claim 6, wherein the first electrical connector includes a first row of electrical contacts on a first face of the first half of the integrated connector, and the second electrical connector includes a second row of electrical contacts that aligns with the first row of electrical contacts when the first half of the integrated connector is mated with the second half of the integrated connector.

13. The bronchoscope of claim 12, wherein the first row of electrical contacts includes a plurality of metal bumps and the second row of electrical contacts includes a plurality of metal recesses to receive the plurality of metal bumps.

14. The bronchoscope of claim 13, wherein:
the handle includes an access door movable between an open position and a closed position; and
the first working channel connector, the first mechanical pulley connector, and the first electrical connector are exposed when the access door is in the open position.

15. A system for performing an endoscopy and an intubation on a patient, the system comprising:
a first working channel connector at a housing;
a first electrical connector at the housing; and
one or more first mechanical pulley connectors at the housing;
a working channel disposed in an insertion cord and having a second working channel connector at an end of the working channel to form a first detachable connection with the first working channel connector;
an electrical wire disposed in the insertion cord having a second electrical connector at an end of the electrical wire to form a second detachable connection with the first electrical connector; and
a mechanical pulley cable disposed in the insertion cord having one or more second mechanical pulley connectors at an end of the mechanical pulley cable to from a third detachable connection with the one or more first mechanical pulley connectors.

16. The system of claim 15, wherein the housing is a handle of a bronchoscope.

17. A device for performing a bronchoscopy on a patient, the device comprising:
a housing including:
a first working channel connector;
a first electrical connector; and
a first mechanical pulley connector; and an insertion cord including:
a second working channel connector at a detachable end of the insertion cord to form a first detachable connection with the first working channel connector;
a second electrical connector at a detachable end of the insertion cord to form a second detachable connection with the first electrical connector; and
a second mechanical pulley connector at a detachable end of the insertion cord to form a third detachable connection with the first mechanical pulley connector.

18. The device of claim 17, wherein engaging a first half of an integrated connector with a second half of the integrated connector causes:
the first working channel connector to mate with the second working channel connector;
the first electrical connector to mate with the second electrical connector; and
the first mechanical pulley connector to mate with the second mechanical pulley connector.

19. The device of claim 18, wherein:
the first mechanical pulley connector includes a recess and the second mechanical pulley connector includes a disc to be received by the recess when the first half of the integrated connector is rotatably engaged with the second half of the integrated connector.

20. The device of claim 17, wherein the first detachable connection, the second detachable connection, and the third detachable connection are made with a one, two, or three engagement actions on a single connector.
